(19) United States
(12) Patent Application Publication
(10) Pub. No.: US 2020/0147242 A1
(43) Pub. Date: May 14, 2020

(54) MAGNETIC, THERMOSENSITIVE, FLUORESCENT MICELLE AND METHOD FOR PREPARING THE SAME

(71) Applicant: Ningxia Medical University, Yinchuan (CN)

(72) Inventors: Guojing GOU, Yinchuan (CN); Min ZHANG, Yinchuan (CN); Xueqin JIN, Yinchuan (CN); Jianhong YANG, Yinchuan (CN); Huiqin YAO, Yinchuan (CN); Ling LI, Yinchuan (CN)

(21) Appl. No.: 16/729,470

(22) Filed: Dec. 29, 2019

Related U.S. Application Data
(63) Continuation-in-part of application No. PCT/CN2019/112616, filed on Oct. 22, 2019.

(30) Foreign Application Priority Data
Oct. 24, 2018 (CN) 201811242354.1

(51) Int. Cl. A61K 49/00; C09K 11/89; C09K 11/02; C08G 81/02; B01J 13/08; B01J 13/22; A61K 41/00; A61K 31/513; A61K 9/107; A61K 47/34

(52) U.S. Cl. CPC A61K 49/0082; C09K 11/892; C09K 11/02; C08G 81/027; B01J 13/08; B82Y 40/00; A61K 49/0067; A61K 41/0052; A61K 31/513; A61K 9/1075; A61K 47/34; B01J 13/22

(57) ABSTRACT

A magnetic, thermosensitive, fluorescent micelle includes a core, a carrier wrapping the core, and a plurality of water-soluble near-infrared CdHgTe quantum dots (QD) disposed on the carrier. The core includes dextran-magnetic layered double hydroxide-fluorouracil (DMF). The carrier includes a tripolymer of poly(N-isopropylacrylamide-co-N,N-dimethylacrylamide)-b-polylactic acid (PLA). N-isopropylacrylamide-co-N,N-dimethylacrylamide of the tripolymer includes a hydrophilic group and a hydrophobic carbon frame. The hydrophilic group is oriented outwards with respect to the and forms a shell. The hydrophobic carbon frame and polylactic acid are restrained to wrap the dextran-magnetic layered double hydroxide-fluorouracil to form the core.

### CROSS-REFERENCE TO RELATED APPLICATIONS

This application is a continuation-in-part of International Patent Application No. PCT/CN2019/112616 with an international filing date of Oct. 22, 2019, designating the United States, now pending, and further claims foreign priority benefits to Chinese Patent Application No. 201811242354.1 filed Oct. 24, 2018. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass. 02142.

### BACKGROUND

The disclosure relates to a magnetic, thermosensitive, fluorescent micelle and a method for preparing the same.

Conventional magnetic, thermosensitive micelles include Fe₃O₄-micelle composites. However, the magnetic carrier Fe₃O₄ is not directly combinable with drugs.

The principle underlying known preparation methods is to mix fluorescent materials with Fe₃O₄ particles or drug molecules, and then to encapsulate them inside a thermosensitive system. However, this leads a relatively low intensity of fluorescence and does not meet the requirements of deep in vivo imaging.

### SUMMARY

The disclosure provides a magnetic, thermosensitive, fluorescent micelle that features near-infrared fluorescence emission, magnetic targeted transport, and thermal controlled release properties.

The magnetic, thermosensitive, fluorescent micelle comprises a core, a carrier wrapping the core, and a plurality of water-soluble near-infrared CdHgTe quantum dots (QD) disposed on the carrier; the core comprises dextran-magnetic layered double hydroxide-fluorouracil (DMF); the carrier comprises a tripolymer of poly(N-isopropylacrylamide-co-N,N-dimethylacrylamide)-b-polylactic acid (PLA); N-isopropylacrylamide-co-N,N-dimethylacrylamide of the tripolymer comprises a hydrophilic group and a hydrophobic carbon frame; the hydrophilic group is oriented outwards with respect to the and forms a shell, and the hydrophobic carbon frame and polylactic acid are restrained to wrap the dextran-magnetic layered double hydroxide-fluorouracil to form the core. The lower critical solution temperature of the magnetic, thermosensitive, fluorescent micelle is equal to or higher than the body temperature.

The lower critical solution temperature of the magnetic, thermosensitive, fluorescent micelle is 42° C.

The monomers for synthesizing the tripolymer are N-isopropylacrylamide (NIPAM), N,N-dimethylacrylamide (DMAM) and lactide.

A method of preparation of the magnetic, thermosensitive, fluorescent micelle comprises:

1) copolymerizing N-isopropylacrylamide and N,N-dimethylacrylamide in the presence of 2,2′-azobis(2-methylpropion amidine) dihydrochloride thereby forming a dipolymer of P(NIPAM-co-DMAM)-OH; polymerizing the dipolymer of P(NIPAM-co-DMAM)-OH and lactide in the presence of stannous octanoate thereby yielding an amphiphilic tripolymer of P(NIPAM-co-DMAM)-b-PLA;

2) combining the amphiphilic tripolymer of P(NIPAM-co-DMAM)-b-PLA and dextran-magnetic layered double hydroxide-fluorouracil to yield a magnetic thermosensitive precursor through synchronous hydration and dialysis;

3) synthesizing water-soluble near-infrared CdHgTe quantum dots by one-pot synthesis in an aqueous phase; and

4) attaching the water-soluble near-infrared CdHgTe quantum dots prepared in 3) to a surface layer of the magnetic thermosensitive precursor prepared in 2) by electrostatic bonding technology.

The dipolymer of P(NIPAM-co-DMAM)-OH is prepared as follows: mixing N-isopropylacrylamide and N,N-dimethylacrylamide in a mass ratio of 95-85:5-15 to form a mixture and dissolving the mixture in an organic solvent A; aerating the organic solvent A with nitrogen to remove oxygen, followed by an addition of the initiator 2,2′-azobis(2-methylpropion amidine) dihydrochloride; 10-12 h later at a constant temperature of 70-80° C., precipitating the resulting product with excess ether, and filtering and drying under vacuum; where the organic solvent A is tetrahydrofuran or chloroform; and the addition amount of the initiator 2,2′-azobis(2-methylpropion amidine) dihydrochloride accounts for 1 to 2 wt. % of that of N-isopropylacrylamide and N,N-dimethylacrylamide.

The amphiphilic tripolymer of P(NIPAM-co-DMAM)-b-PLA is prepared as follows: mixing the dipolymer of P(NIPAM-co-DMAM)-OH with the lactide in a mass ratio of 40-30:60-70 to form a mixture; dissolving the mixture in an organic solvent B, followed by an addition of the catalyst stannous octanoate; aerating the organic solvent B with nitrogen to remove oxygen, reacting at a constant temperature of 120-140° C. for 24-28 h, and precipitating the resulting product with excess ether, and drying under vacuum; where the organic solvent B is anhydrous xylene or toluene.

Combining the amphiphilic tripolymer of P(NIPAM-co-DMAM)-b-PLA and dextran-magnetic layered double hydroxide-fluorouracil comprises: dissolving the dextran-magnetic layered double hydroxide-fluorouracil and the tripolymer with an organic solvent N,N-dimethylformamide; transferring the resulting mixture to a dialysis bag, and dialyzing against distilled water with vigorous stirring at room temperature; the molecular-weight cut-off value of the dialysis bag is 8000-14000 g·mol⁻¹; the dialysis is continued for 48 h; the distilled water is renewed every 1 h for the first 5 h, and then every 12 h; the mass ratio of the dextran-magnetic layered double hydroxide-fluorouracil to the tripolymer is 5-20:20.

Attaching the water-soluble near-infrared CdHgTe quantum dots to the surface layer of the magnetic, thermosensitive micelle comprises:

a. mixing and grinding the water-soluble near-infrared CdHgTe quantum dots and the magnetic, thermosensitive micelle in a mass ratio of 1-3:1-1 to prepare a mixed powder; and

b. suspending and dispersing the mixed powder in absolute ethanol, and ultrasonically dispersing the suspension; separating the magnetic particles from the mixture using a magnet, followed by centrifuging; washing the resulting product with absolute ethanol, and then drying under vacuum; where the ultrasonic dispersing comprises oscillating the suspension in an ultrasonic water bath at 30-50° C. for 1 to 3 h; the magnet attraction comprises adsorbing magnetic solid materials from the suspension by a magnet after ultrasonic dispersion, and discarding the liquid-phase to remove the unbound CdHgTe quantum dots; washing the solid-phase sample 2-3 times with anhydrous ethanol; the vacuum drying are performed at 50-60° C. and 0.085 megapascal.

The method for preparing water-soluble near-infrared CdHgTe quantum dots (QD) is described as follows:

i) dissolving Cd(NO₃)₂ with water until the solution clarified, removing oxygen at room temperature and magnetic stirring at 300 rpm under nitrogen protection; adding Hg(NO₃)₂ solution in a molar ratio of Cd²⁺/Hg²⁺=1:0.03-0.06 to the clarified solution, and uniformly dispersing the mixture; adding mercaptopropionic acid solution in a molar ratio of Cd²⁺/Hg²⁺=1:0.03-0.06 to the resulting dispersion for in-situ reaction; and adjusting the pH of the resulting mixture to 6.0-9.0, to yield a precursor solution of Cd²⁺—Hg²⁺-mercaptopropionic acid;

ii) weighting tellurium (Te) powder and NaBH₄ solid in a molar ratio of Te/NaBH₄=1:1.8-2.2, and dissolving the mixed powder with water; magnetic stirring the mixture under nitrogen protection at 50° C. and 300 rpm until Te powder disappears, to yield NaHTe slurry;

iii) adding the NaHTe slurry prepared in ii) in a molar ratio Cd²⁺/NaHTe=1:0.10-0.130 to the precursor solution of Cd²⁺—Hg²⁺-mercaptopropionic acid prepared in i); magnetic stirring the mixed slurry at 300 rpm at a constant temperature under nitrogen protection until fluorescence intensity of the liquid phase no longer increases; and

iv) aging the stirred slurry, adding ethanol to settle and separate the slurry; discarding the supernatant, and centrifuging at 5000 rpm at room temperature; washing the obtained solid-phase sample with absolute ethanol and drying under vacuum.

In i), the time for removing oxygen is 30-45 min; the in-situ reaction time is 30-60 min; and the pH is adjusted with 2.0 mol/L NaOH. In iv), the time for aging of the stirred slurry is 50-70 min; washing the resulting product with absolute ethanol 2-3 times; the vacuum drying is performed at 65-80° C. and 0.085 megapascal.

The disclosure employs N-isopropylacrylamide (NIPAM), N,N-dimethylacrylamide (DMAM) and lactic acid (LA) to synthesize a tripolymer of P(NIPAM-co-DMAM)-b-PLA, a thermosensitive polymer (TRM). The magnetic, sustained release drug delivery system of dextran-magnetic layered double hydroxide-fluorouracil (DMF) constitutes the core of thermosensitive micelles, to prepare a magnetic, thermosensitive micelle (P(NIPAM-co-DMAM)-b-PLA-DMF) with magnetic response and temperature sensitivity. The water-soluble near-infrared CdHgTe quantum dots are attached to the surface layer of the magnetic, thermosensitive micelles by electrostatic bonding technology, thereby yielding DMF-TRM-QD fluorescent micelles with near-infrared fluorescence emission, magnetic targeted transport, and thermal controlled release properties. The produced micelles can be quickly transported into the cells, and emit red fluorescence in the cells after being stimulated; the cell internalization and transport efficiency of the micelles increase linearly with magnetic field strength, and the density of the cell nucleus and the degree of pyknosis also change regularly with magnetic field gradient, which makes it possible to regulate the effect of cell imaging and chemotherapy by the applied magnetic field strength and the direction of action. Therefore, the method is of great special application value in in vivo diagnostic imaging and cancer treatment.

The hydrophilic groups of the block copolymer are oriented outward towards water to form a micellar shell. The hydrophobic carbon frame and the PLA block are restrained to wrap the core comprising the dextran-magnetic layered double hydroxide-fluorouracil to form a core-shell type micelle structure; the lower critical solution temperature (LCST) is 42° C., which is much higher than the LCST (32° C.) of the polymerization of (N-isopropyl acryl), and higher than the physiological temperature of the human body (37° C.), but lower than the temperature of tumor tissues. Therefore, the polymer can exist stably under normal physiological conditions, and circulates in the body for a long time and is not easy to be cleared from the body. The polymer can be precipitated and highly concentrated in the heated part of the tumor, which achieves the purpose of heat-induced targeted therapy and is suitable for the application requirements of drug transport in vivo. The polymer has a lower critical micelle concentration (7.413 μg·mL⁻¹), with a higher sol stability and sensitive magnetic responsiveness. The light transmittance and particle size of micelles show significant thermo-sensitivity. The release of the drug presents different kinetic models before or after phase transition. And the drug release rate and cumulative release increase with temperature.

In summary, the disclosure employs DMF as the core of a thermosensitive micelle to solve the problems of drug encapsulation, targeted transport, and heat shock release. Water-soluble near-infrared quantum dots are combined into the micellar shell with DMF-based electrostatic radiation, which solves the problem of biological transport imaging of magnetic, thermosensitive micelles. The synthesized thermosensitive carrier P(NIPAM-co-DMAM)-b-PLA has a proper LCST, and the cost is low; it also has high drug loading rate, good water solubility, and high sol stability. The micelles have ideal thermosensitive properties, magnetic targeted transport, fluorescence tracing functions, and exhibit special application prospects in magnetic-thermal double targeting chemotherapy for tumors.

### BRIEF DESCRIPTION OF DRAWINGS

FIG. 1 shows morphology of DMF-TRM micelles (A) under transmission electron microscope and the fluorescence image of DMF-TRM-QD micelles (B);

FIG. 2 shows imaging results of MGC-803 cells after incubation with DMF-TRM-QD complex for 1 h (A), 3 h (B), 5 h (C) and 7 h (D); (wherein green fluorescence represents the nuclear signal of Hoechst 33342 labeled cells, red fluorescence represents the signal of DMF-TRM-QD labeled cells, yellow fluorescence represents overlap of these two colors, which are observed with a 40× objective lens);

FIG. 3 shows confocal imaging results of MGC-803 cells after incubation with DMF-TRM-QD under hyperthermia at 42° C. and different applied magnetic field gradient (the number of magnets are respectively 0, 5, 10, 15, 20, 25); (where green fluorescence represents the nuclear signal Hoechst 33342 labeled cells; red fluorescence represents the signal of fluorescent micelle particles in the cells; yellow fluorescence represents overlap of these two colors);

FIG. 4 shows linear relationship between the average optical density of red fluorescence in a cell and the applied magnetic field strength (T).

### DETAILED DESCRIPTION OF EMBODIMENTS

The reagents used in the chemical synthesis of the invention are all conventional commercial reagents, and the materials used in the biological experiment are all commercial products. It should be noted that the following embodiments are intended to describe and not to limit the disclosure.

### EXAMPLE 1

### Synthesis of DMF-TRM-QD Micelles

### 1) Preparation of P(NIPAM-co-DMAM)-b-PLA tripolymer (poly(N isopropylacrylamide-co-N,N dimethylacrylamide)-b-polylactic acid)

0.41 g of NIPAM and 0.046 g of DMAM were weighed as raw materials according to the mass ratio of NIPAM/DMAM=90:10, and they were mixed and put into a three-necked flask. 25 mL of freshly distilled tetrahydrofuran (THF) was added to dissolve the mixture to form a solution. 0.009 g of copolymerization initiator AMAD was then added to react with the solution at a constant temperature of 80° C. for 10 h after oxygen was removed under nitrogen protection for 30 min. The resulting product was precipitated with excess ether, filtered under vacuum, and dried under vacuum at room temperature for 12 h to obtain P(NIPAM-co-DMAM)-OH dipolymer (poly(N isopropylacrylamide)-co-N,N dimethylacrylamide)). Then the P(NIPAM-co-DMAM)-OH dipolymer was precipitated with excess ether, filtered under vacuum, and dried under vacuum at room temperature for 12 h to obtain a pure solid-phase dimer.

0.1879 g of lactide and 0.0909 g of dimer powder were weighed according to a mass ratio of D,L-lactide/P(NIPAM-co-DMAM)-OH=67:33, and they were mixed and placed into a three-necked flask. 20 mL of anhydrous xylene were added and stirred to dissolve the mixture. After 2-3 drops of stannous octoate were added and oxygen was removed under nitrogen protection for 30 min, the solution reacted at a constant temperature of 135° C. for 24 h to obtain P(NIPAM-co-DMAM)-b-PLA tripolymer (poly(N isopropylacrylamide-co-N,N dimethylacrylamide)-b-polylactic acid). The P(NIPAM-co-DMAM)-b-PLA tripolymer was precipitated with excess diethyl ether and dried to constant weight under vacuum at 30° C. for 48 h to obtain a pure solid-phase thermosensitive tripolymer.

### 2) Synthesis of Magnetic and Thermosensitive Micelles (DMF-TRM)

20 mg of DMF powder and 20 mg of P(NIPAM-co-DMAM)-b-PLA tripolymer were weighed and dissolved with 10 mL of N,N-dimethylformamide to form a solution. The solution was transferred into a dialysis bag and dialyzed against 1000 mL of distilled water at room temperature with vigorous stirring for 24 h. The dialysis medium is replaced every 1 h for the first 5 h, and then every 12 h. A thermosensitive micelle solution was obtained after 2 days of dialysis, and then placed into a clean 100-mL beaker. After coagulating at −20° C., the micelle solution was quickly transferred to a pre-cooled vacuum freeze dryer to raise the temperature for preparation of micellar lyophilized powder which was stored at 4° C.

### 3) Synthesis of Water-Soluble Near-Infrared CdHgTe Quantum Dots

5.3211 g of Cd(NO₃)₂.4H₂O solids were weighed and dissolved in excess water to give a final volume of 100 mL. 10 mL of the solution was transferred into a 1000 mL reactor, followed by dilution with 900 mL of water. The oxygen was removed with magnetic stirring at 300 rpm under nitrogen protection at room temperature. Then 5 μL of Hg(NO₃)₂.4H₂O saturated solution was added and stirred for 30 min, followed by addition of 0.00517 mol of mercaptopropionic acid (MPA). 2.0 mol/L NaOH solution was then added dropwise under magnetic stirring at 300 rpm, and the pH value of the slurry was adjusted to 7.0 to yield a Cd²⁺—Hg²⁺-MPA precursor.

0.27 g Te powder and 0.16 g of solid-phase NaBH₄ were weighed and placed into a 100 mL reactor, followed by addition of 10 mL of double-distilled water. The solution was magnetic stirred at 300 rpm in the presence of nitrogen at a constant temperature of 50° C. until Te powder disappeared, thus yielding NaHTe. The prepared slurry was injected into the precursor solution of Cd²⁻—Hg²⁺-mercaptopropionic acid, and the mixed slurry was magnetic stirred at 300 rpm at a constant temperature of 50° C. under nitrogen protection until fluorescence intensity of the liquid phase no longer increased. After static aging of the stirred slurry, ethanol was added to settle and separate; the supernatant was discarded, and the precipitate was centrifuged at 5000 rpm at room temperature; the obtained solid-phase sample was washed with absolute ethanol 2-3 times and dried in a vacuum dryer at 65° C. and 0.085 megapascal.

### 4) Synthesis of DMF-TRM-QD Fluorescent Micelles by the Use of DMF-TRM Micellar Powder and Water-Soluble Near-Infrared CdHgTe Quantum Dots Via Electrostatic Bonding:

a. A certain amount of water-soluble near-infrared CdHgTe quantum dots and DMF-TRM micellar powder were weighed according to the mass ratio of QD:DMF-TRM=1:1, they were mixed and ground in an agate mortar for 10-50 min;

b. the mixed powder prepared in a) was suspended and dispersed with absolute ethanol, and the suspension was placed in a water bath at 30-50° C. and ultrasonically dispersed for 2 h;

c. the magnetic solid substance in the liquid-phase was attracted to a magnet, and the liquid-phase was discarded to remove the unbound CdHgTe quantum dots. The processes of dispersing, ultrasonic, and magnetic separation of the magnetic solid substance were repeated. Then the separated solid was centrifuged at 5000 rpm at room temperature, and the solid-phase was washed with anhydrous ethanol 2-3 times and dried in a vacuum dryer at 50-60° C. and 0.085 megapascal, thus yielding the final product.

### EXAMPLE 2

### Synthesis of DMF-TRM-QD Micelles

### 1) Preparation of P(NIPAM-co-DMAM)-b-PLA tripolymer (poly(N isopropylacrylamide-co-N,N dimethylacrylamide)-b-polylactic acid)

0.41 g of NIPAM and 0.046 g of DMAM were weighed as raw materials according to the mass ratio of NIPAM/DMAM=90:10, and they were placed into a three-necked flask. 25 mL of freshly distilled THF was added to dissolve them to form a solution. 0.009 g of copolymerization initiator AMAD was added to react with the solution at a constant temperature of 80° C. for 10 h after oxygen was removed under nitrogen protection for 30 min, thus yielding P(NIPAM-co-DMAM)-OH) dipolymer (poly(N-isopropylacrylamide-co-N,N-dimethylacrylamide)). Then, P(NIPAM-co-DMAM)-OH dipolymer was precipitated with excess ether, filtered under vacuum, and dried under vacuum at room temperature for 12 h to obtain a pure solid-phase dimer.

0.20 g of lactide and 0.13 g of dimer powder were weighed according to a mass ratio of D,L-lactide/P(NIPAM-co-DMAM)-OH=67:33, and they were mixed and placed into a three-necked flask. 20 mL of anhydrous xylene were added and stirred to dissolve the mixture; after 2-3 drops of stannous octoate were added and oxygen was removed under nitrogen protection for 30 min, the solution reacted at a constant temperature of 135° C. for 24 h to yield P(NIPAM-co-DMAM)-b-PLA tripolymer (poly(N isopropylacrylamide-co-N,N dimethylacrylamide)-b-polylactic acid). The P(NIPAM-co-DMAM)-b-PLA tripolymer was precipitated with excess diethyl ether and dried to constant weight under vacuum at 30° C. for 48 h to obtain a pure solid-phase tripolymer.

### 2) Preparation of Magnetic, Thermosensitive Micelles (DMF-TRM)

5 mg DMF powder and 20 mg of lyophilized powder of thermosensitive polymer were weighed to prepared a DMF-TRM micellar lyophilized powder according to 2) in Example 1.

### 3) Preparation of Water-Soluble Near-Infrared CdHgTe Quantum Dots

The method is the same as 1) in Example 1.

### 4) Preparing of DMF-TRM-QD Fluorescent Micelles by the Use of DMF-TRM Micellar Powder and Water-Soluble Near-Infrared CdHgTe Quantum Dots Via Electrostatic Bonding

a. 8 mg of water-soluble near-infrared CdHgTe quantum dots and DMF-TRM micellar powder were weighed according to the mass ratio of QD:DMF-TRM=1:3, they were mixed and ground in an agate mortar for 10-50 min;

b. the mixed powder prepared in a) was suspended and dispersed with absolute ethanol, and the suspension was placed in a water bath at 30-50° C. and ultrasonically dispersed for 2 h;

c. the magnetic solid substance in the liquid-phase was attracted to a magnet, and the liquid-phase was discarded. The processes of dispersing, ultrasonic, and magnetic separation of the magnetic solid substance were repeated. Then the separated solid was centrifuged at 5000 rpm at room temperature, and the solid-phase was washed with anhydrous ethanol 2-3 times and dried in a vacuum dryer at 50-60° C. and 0.085 megapascal, thus yielding the final product.

### EXAMPLE 3

### Synthesis of DMF-TRM-QD Micelles

### 1) Preparation of P(NIPAM-co-DMAM)-b-PLA tripolymer (poly(N isopropylacrylamide-co-N,N dimethylacrylamide)-b-polylactic acid).

0.6 g of NIPAM and 0.032 g of DMAM were weighed as raw materials according to the mass ratio of NIPAM/DMAM=95:5. And P(NIPAM-co-DMAM)-b-PLA tripolymer was prepared according to the technical conditions and process of Example 1.

### 2) Preparation of Magnetic, Thermosensitive Micelles (DMF-TRM)

15 mg of DMF powder and 20 mg of lyophilized powder of thermosensitive polymer were weighed and dissolved with 10 mL of N,N-dimethylformamide. The resulting mixture was transferred into a dialysis bag, and dialyzed against 1000 mL of distilled water at room temperature with vigorous stirring for 48 h. The distilled water was replaced every 1 h for the first 5 h, and then every 12 h. The thermosensitive micellar solution was obtained after 46 h of dialysis, and then placed into a clean 100-mL beaker. After coagulating at −20° C., the micelle solution was quickly transferred to a pre-cooled vacuum freeze dryer to increase the temperature for preparation of micellar lyophilized powders.

### 3) Preparation of Water-Soluble Near-Infrared CdHgTe Quantum Dots

The method is the same as 1) in Example 1.

### 4) Preparation of DMF-TRM-QD Fluorescent Micelles by the Use of DMF-TRM Micellar Powder and Water-Soluble Near-Infrared CdHgTe Quantum Dots Via Electrostatic Bonding

a. 16 mg of water-soluble near-infrared CdHgTe quantum dots and 32 mg of DMF-TRM micellar powder were weighed according to the mass ratio of QD:DMF-TRM=1:3, then they were mixed and ground in an agate mortar for 10-50 min;

b. the mixed powder prepared in a) was suspended and dispersed with absolute ethanol, and the suspension was placed in a water bath at 30-50° C. and ultrasonically dispersed for 2 h;

c. the magnetic solid substance in the liquid-phase was attracted to a magnet, and the liquid phase was discarded. The processes of dispersing, ultrasonic, and magnetic separation of the magnetic solid substance were repeated. Then the separated solid was centrifuged at 5000 rpm at room temperature, and the solid-phase was washed with anhydrous ethanol 2-3 times and dried in a vacuum dryer at 50-60° C. and 0.085 megapascal, to yield the final product.

Evaluation of the Implementation and Effect

The laser-scanning fluorescence confocal imaging technology was employed to verify the effects of cell transport and biological imaging of the DMF-TRM-QD fluorescent micelles. The experimental results showed that the fluorescent micelles formed by the combination of DMF magnetic, thermosensitive micelles and biological quantum dots, which can enter cells and reach nucleus regions. The micelles showed good fluorescent labeling and performance of magnetic and thermal targeting, and has great application prospects in magnetic and thermal targeted chemotherapy for tumors.

Experimental Process and Results

(1) Morphological Features of DMF-TRM-QD Fluorescent Micelles.

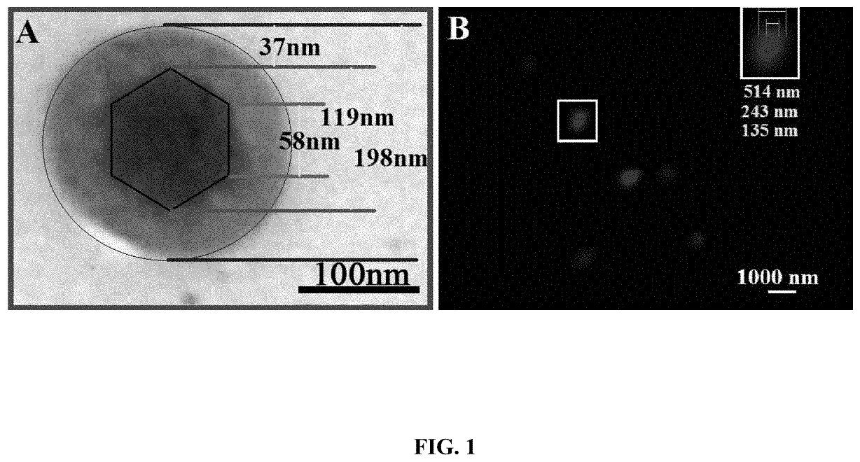

The morphological features of DMF-TRM micelles was determined by a transmission electron microscope (TEM, H-7560B, Hitachi, Tokyo). Two drops of micellar solution were added on the copper mesh, dried naturally at room temperature, and observed and photographed with an electron microscope under an accelerated voltage of 80 kV. As shown in the image A of FIG. 1, the dark hexagonal-layered appearance, which were located in the nuclear layer of micelles, are DMF nanoparticles with a side length of 58 nm. The diameter of internal cavity is 119 nm, with a shell thickness of 37 nm, and the micelle diameter is 198 nm. The morphological features of micelles demonstrated that the hexagonal magnetic DMF particles play a crucial guiding role in forming the core-shell structure of the complex micelles. The rigid magnetic DMF particles play a supporting role in the structure of the micelle cavity, and the hydroxyl groups on the surface of the DMF particles can form hydrogen bonds with the hydration layer of hydrophobic groups of the micelles, which make the core-shell structure of the micelles more contractive, stable and highly dispersible.

Quantum dot marker can be used for tracking the transport trajectory of DMF thermo-responsive micelles (TRM). The image B of FIG. 1 is a fluorescence image of DMF-TRM-QD complex particles observed and photographed with a 100× oil immersion objective lens. The prepared CdHgTe quantum dots with sizes below 10 nm, can emit red fluorescence which was excited by a 488 nm green helium-neon laser. The DMF-TRM-QD complex particles had a particle size of about 500 nm in aqueous solution at room temperature. As shown in the image B of FIG. 1, the spherical micelles had an external diameter of about 514 nm, which confirmed the size of the DMF-TRM-QD micelles.

The DMF particles, which comprised a plurality of hydroxyl groups on the surface thereof, were wrapped in the micelle core and capable of combining with QD. Therefore, the DMF-loaded micelles had a high fluorescence intensity in the micelle core, and an internal diameter of about 243 nm, which illustrated that the aggregated DMF particles were wrapped in the thermosensitive micelles. There were also a large number of hydrophilic groups on the surface of the composite micelles, with a shell thickness of about 135 nm, and the shell had a weak fluorescence intensity, which can reflect the thickness of the hydration layer of the micelles. The above results demonstrated that DMF-loaded micelles had combined successfully with QD to form a fluorescent complex.

(2) Cell Transport and Biological Imaging of DMF-TRM-QD Fluorescent Micelles

MGC-803 cells in exponential phase were digested with trypsin, and centrifuged to make a single cell suspension. The cells were seeded at 3×105 cells per well in 6-well cell culture plates and cultured for 24 h until the cells adhere to the wall of the 6-well plates. The supernatant of the culture medium in each well was discarded and the cells were washed with PBS 3 times. 2 mL of drug-containing medium was added to each well. The room-temperature groups were taken out of the cell culture plates after incubation for 1 h, 3 h, 5 h, and 7 h, respectively. The hyperthermia groups were incubated in a constant-temperature box at 42° C. for 30 min and then quickly transferred to an incubator at 37° C. for 0.5 h, 2.5 h, 4.5 h, and 6.5 h, respectively. All the supernatant was aspirated carefully and discarded. After the cells were washed with PBS three times, 1 mL of Hoechst 33342 (10 μg·mL⁻¹, nucleating agent) was added and incubated for 30 min. Once again, all the supernatant was aspirated carefully and discarded, and then the cells were washed with PBS three times. 1 mL of 4% paraformaldehyde was added per well to fix the cells for 5 min and then washed with PBS three times. The cover slips were gently lifted with the tip of a burned syringe needle and removed quickly with a tweezer. The excess liquid on the slides was removed with filter paper, followed by an addition of 1-2 drops of anti-fade mounting medium. Then the mounted slides can be store at −20° C., protected from light. In the confocal imaging experiment, the cells in the field of view were observed with a 40× objective lens; the fluorescent micelles were observed with a 100× oil immersion objective lens; the fluorescence of QD was excited with a 488 nm green helium-neon laser; the emission wavelength was detected with a 560-660 nm bandpass filter. The excitation wavelength was 405 nm, and the fluorescence of the nuclear dye Hoechst 33342 was detected with a430-460 nm bandpass filter.

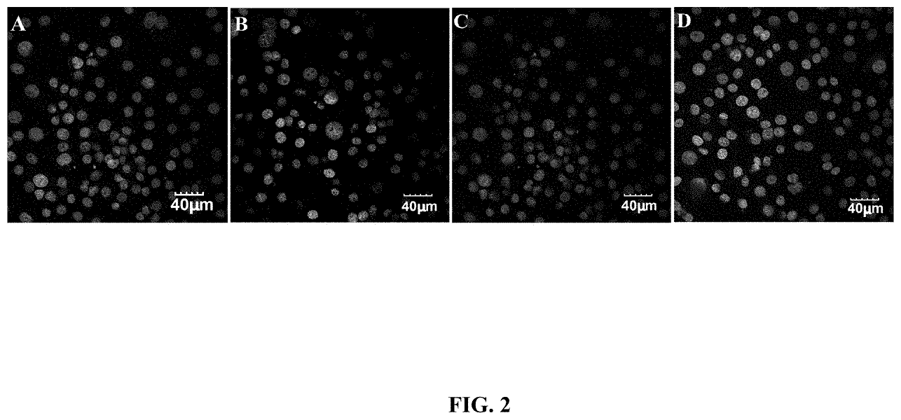

FIG. 2 showed the confocal microscopy fluorescence images after incubating human gastric cancer MGC-803 cells with DMF-TRM-QD micelles. The nucleus of the cells was stained with Hoechst 33342 to emit green fluorescence, but the DMF-TRM-QD micelles entered the cells and emitted red fluorescence inside the cells. The green fluorescence and the red fluorescence overlapped to yield yellow fluorescence. The image A of FIG. 2 confirmed that the nucleus had regular morphology and uniform size after the micelles interacted with cells for 1 h. But the red fluorescence was very weak in the cells, and only a few cells expressed the red fluorescence outside or near the nucleus. The results demonstrated that the fluorescent micelles had a low degree of cell internalization, and no significant effect on cell viability for the fluorescent micelle particles carrying nano-drugs. After the cells were incubated with the fluorescent complex for 3 h, the number of fluorescent micelles entering the cells was significantly increased relative to that after the incubation for 1 h, which were analyzed from the red fluorescence distribution. With regard to the nucleus morphology, the diameter distribution of the nucleus began to differentiate. Some nucleuses tended to be swelled and rounded, and the other nucleuses exhibited reduction in size and darkened in color. The results showed that after interaction for 3 h, the fluorescent micelles appeared in the central part of some nucleuses, which demonstrated that the cell internalization had reached significant level and the drug carried by the micelles also started to play an intervention role. The nucleus swelling may be related to the comprehensive biochemical response of the DMF-loaded micelles and the biological substances that constitute the organelles and nucleus, more than the effect of the released of the FU drugs. The nucleus pyknosis illustrated that the biochemical reactions between DMF-loaded micelles and nuclear materials may have terminated. After incubation for 5 h, there was no significant increase in number of the red fluorescent spots emitted from the cells compared with the image B of FIG. 2. However, the phenomenon still cannot determine whether the degree of cell internalization of fluorescent micelles increase with time. When the fluorescent micelles entered the cells in a smaller size and a more dispersed state, the imaging effect can be influenced by the imaging angle, organelle fusion, masking, etc. These questions caused the difficulty in clearly displaying the degree of cell internalization. The comparison of the hue in the pictures showed that the yellow component of the nucleus was significantly increased compared to that in the image B of FIG. 2, especially the yellow component of the nucleolus increased significantly, illustrating that the red fluorescent in the nucleus showed an increasing trend over time. With regard to the morphology of the nucleus, the increase in the density of the nucleus and the degree of nucleus pyknosis confirmed that the pharmacological effect of the fluorescent micelles in the cells became manifest over time. The image D of FIG. 2 showed that the changes in the state of aggregation and survival of the nucleus caused by fluorescent micelles are completely similar to those in the image C of FIG. 2. Although the number of macroscopically visible red aggregate particles decreased, the degree of cell coloration increased, which conformed that the small-sized and dispersed fluorescent micelles had increased in degree of cell internalization. The above results indicated that it takes just under 1 h for the fluorescent magnetic micelles of the disclosure to complete the process of cell internalization kinetics. The micelles reached the nucleus and induced the obvious differentiation of the nucleus morphology after administration for 3 h, and the pharmaceutical effect gradually increased with the prolonged action of the time.

The number of DMF-TRM-QD micelles entering the cell can be indirectly determined by calculating the average optical density of red fluorescence in the cell imaging. For four experimental groups carried out at normal temperature for 1 h, 3 h, 5 h and 7 h, respectively, the average optical density value (see Table 1) of red fluorescence was calculated with Image-Pro Plus analysis software. And a correlation analysis was performed to reveal the interrelationship between the length of intervention time and the average optical density value. Specifically, the analysis results were fitted to the following linear equation: Dmean=0.0409 t-0.0465 (where Dmean represented the average optical density value of red fluorescence emitted from the cells, t represented the intervention time of fluorescence complex, unit: hour). The correlation coefficient was 0.92, illustrating that the number of DMF-TRM-QD micelles entering the cell showed a linear growth trend over time. The confocal microscopy fluorescence images showed that the cell transport and pharmacodynamic process of the DMF-TRM-QD micelles increased with the intervention time, and the number of nano-scale fluorescent micelles wrapped by the cells also increased. The particles being transported to the nucleolus of the cells, can interact with nuclear biological substances to cause the swelling, contraction and solidification of the nucleus, thereby achieving the purpose of eliminating cancer cells and tumor tissues.

TABLE 1
Dmean of different intervention times (n = 3)

| t (h) | 1 | 3 | 5 | 7 |
|---|---|---|---|---|
| Dₘₑₐₙ | 0.0089 ± | 0.0186 ± | 0.0092 ± | 0.1293 ± |
| (X̅ ± s) | 0.0053 | 0.0009 | 0.0004 | 0.0980 |

(3) Effect of Applied Magnetic Responsiveness on Cell Transmission Efficiency of DMF-TRM-QD Fluorescent Micelles.

To investigate the effect of applied magnetic field on cell transmission efficiency of DMF-TRM-QD cells, the confocal imaging was photographed for MGC-803 cells after incubation with DMF-TRM-QD under hyperthermia at 42° C. for 7 h and different gradient of applied magnetic field.

The small sterilized magnets were stuck to the bottom of the external surface of the 6 well cell plates with a sterile white tape prior to the experiments. The clean cover slips were placed at the bottom of the wells. MGC-803 cells were inoculated in the exponential phase and cultured for 24 h until the cells adhere to the wall of the 6-well plates. The supernatant of the culture medium in each well was discarded and the cells were washed with PBS 3 times. 2 mL of drug-containing medium was added to each well. The room-temperature groups were taken out of the cell culture plate after incubation for 1 h, 3 h, 5 h, and 7 h, respectively. The hyperthermia groups were incubated in a constant-temperature box at 42° C. for 30 min and then quickly transferred to an incubator at 37° C. for 0.5 h, 2.5 h, 4.5 h, and 6.5 h, respectively. Subsequently, the processes for mounting and confocal imaging were as the same as in (2).

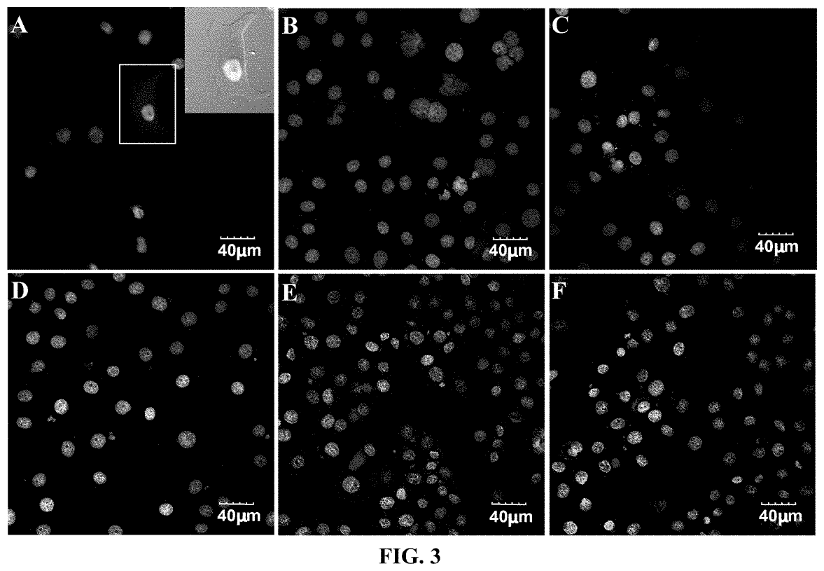

The cells imaging results were shown in FIG. 3. The image A of FIG. 3 showed when there is no magnetic field hyperthermia, only a few cells can be observed with a red fluorescent signal. A brightly dyed cell was observed and the entire cell was red and had a clear cell outline. Fluorescent magnetic micelles reached the nucleolus of the nucleus, and then quantum dots fluoresced and illuminated all areas within the cell at the moment when the cell was fixed and “blackened”. As shown in the images B-E of FIG. 3, the number of cells emitting red fluorescence increased significantly with increasing magnetic field strength, and the fluorescence density increased accordingly.

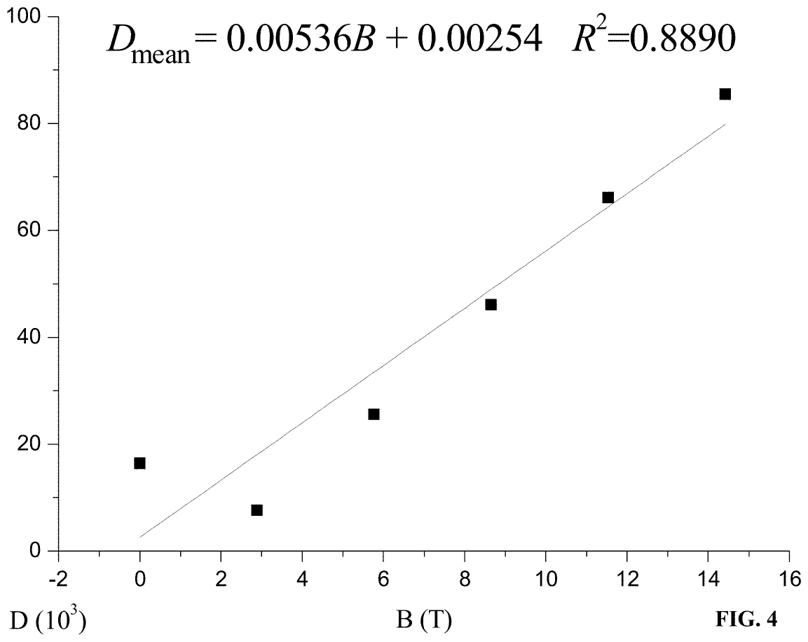

Table 2 showed the average optical density of the red fluorescence in six hyperthermia experimental groups at a steady state temperature of 42° C. And the six magnetic field gradients were designed by Image-Pro Plus software. For the magnets the number were 0, 5, 10, 15, 20 and 25, respectively. For each micro magnetic the strength of was 5770 Gauss or 0.577 T. FIG. 4 showed the linear relationship between the intracellular mean fluorescence density and the applied magnetic field gradient, which fitted the linear equation: Dmean=0.00536 B+0.00254 (Dmean represented the average optical density of red fluorescence emitted from the cells; B represented the applied magnetic field strength; the unit for the magnetic field strength was Tesla T; and the linear correlation coefficient is 0.91). The results showed that the amount of DMF-TRM-QD entering the cell goes up linearly with the magnetic field gradient, which illustrated the intracellular magnetic targeting transport kinetics of fluorescent magnetic micelle particles.

As shown in the images A-F of FIG. 3, the relationship between the nuclear density N and the applied magnetic field gradient fitted the linear equation: N=2.6457 n+20.429 and R2=0.6528, indicating that the nuclear density N was also regularly distributed with the magnetic field gradient.

TABLE 2
Dmean of different magnetic field strength (X̅ ± s)

| (T) | 0 | 2.885 | 5.77 | 8.655 | 11.54 | 14.425 |
|---|---|---|---|---|---|---|
| Dₘₑₐₙ | 0.0164 ± 0.0064 | 0.0077 ± 0.0007 | 0.0256 ± 0.0074 | 0.0461 ± 0.0009 | 0.0662 ± 0.0343 | 0.0855 ± 0.0135 |

The above results showed that DMF-TRM-QD micelles were transported into cells soon after contacting the cells. The micelles were excited by laser beam and emitted a specific wavelength of red fluorescence in cells. An increase in the degree of cell internalization of micelles and the magnetic response of nuclear distribution were achieved by exposing the cells to the applied magnetic fields. Therefore, it was confirmed that the DMF-TRM-QD micelles had special application value for in vivo diagnostic imaging and cancer treatment.

The combination of DMF-loaded micelles and the biological quantum dots can produce DMF-TRM-QD composite particles with multiple functions of fluorescent labeling, thermal sensitivity and magnetic targeting. The cell internalization and transport efficiency of DMF-TRM-QD micelles goes up linearly with the magnetic field strength. The density of nucleus and the degree of pyknosis both showed a regular change with the magnetic field gradient, which made it was possible to improve the effects of intracellular imaging and chemotherapy only by the use of magnetic fields. The successful recombination of DMF-TRM with biological quantum dots and the ideal effect of cell transport demonstrated that DMF-TRM-QD micelles would be an intelligent and multifunctional nano-system with great development prospects and application value.

It will be obvious to those skilled in the art that changes and modifications may be made, and therefore, the aim in the appended claims is to cover all such changes and modifications.

What is claimed is:

1. A micelle, comprising:
1) a core, the core comprising dextran-magnetic layered double hydroxide-fluorouracil (DMF);
2) a carrier wrapping the core, the carrier comprises a tripolymer of poly(N-isopropylacrylamide-co-N,N-dimethylacrylamide)-b-polylactic acid; and
3) a plurality of water-soluble near-infrared CdHgTe quantum dots (QD) disposed on the carrier;
wherein:
N-isopropylacrylamide-co-N,N-dimethylacrylamide of the tripolymer comprises a hydrophilic group and a hydrophobic carbon frame;
the hydrophilic group is oriented outwards with respect to the and forms a shell, and the plurality of water-soluble near-infrared CdHgTe quantum dots is bonded to the shell; and
the hydrophobic carbon frame and polylactic acid are restrained to wrap the core.

2. The micelle of claim 1, wherein a lower critical solution temperature of the micelle is 42° C.

3. The micelle of claim 1, wherein monomers for synthesizing the tripolymer are N-isopropylacrylamide (NIPAM), N,N-dimethylacrylamide (DMAM) and lactide (PLA).

4. A method of preparation of the micelle of claim 1, the method comprising:
1) copolymerizing N-isopropylacrylamide and N,N-dimethylacrylamide in the presence of 2,2′-azobis(2-methylpropion amidine) dihydrochloride thereby forming a dipolymer of P(NIPAM-co-DMAM)-OH; polymerizing the dipolymer and lactide in the presence of stannous octanoate thereby yielding an amphiphilic tripolymer of P(NIPAM-co-DMAM)-b-PLA;
2) synthesizing dextran-magnetic layered double hydroxide-fluorouracil, and combining the amphiphilic tripolymer and the dextran-magnetic layered double hydroxide-fluorouracil to yield a magnetic thermosensitive precursor through synchronous hydration and dialysis;
3) synthesizing water-soluble near-infrared CdHgTe quantum dots by one-pot synthesis in an aqueous phase; and
4) attaching the water-soluble near-infrared CdHgTe quantum dots prepared in 3) to a surface layer of the magnetic thermosensitive precursor prepared in 2) by electrostatic bonding technology.

5. The method of claim 4, wherein preparing the dipolymer of P(NIPAM-co-DMAM)-OH comprises: mixing N-isopropylacrylamide and N,N-dimethylacrylamide in a mass ratio of 95-85:5-15 to form a mixture; dissolving the mixture in an organic solvent A; aerating the organic solvent A with nitrogen to remove oxygen, followed by an addition of 2,2′-azobis(2-methylpropion amidine) dihydrochloride as an initiator; 10-12 h later at a constant temperature of 70-80° C., precipitating a resulting product with excess ether, filtering and drying the resulting product under vacuum.

6. The method of claim 5, wherein the organic solvent A is tetrahydrofuran or chloroform; and an addition amount of the 2,2′-azobis(2-methylpropion amidine) dihydrochloride accounts for 1 to 2 wt. % of that of the N-isopropylacrylamide and N,N-dimethylacrylamide.

7. The method of claim 4, wherein preparing the amphiphilic tripolymer of P(NIPAM-co-DMAM)-b-PLA comprises: mixing the dipolymer of P(NIPAM-co-DMAM)-OH with the lactide in a mass ratio of 40-30:60-70 to form a mixture; dissolving the mixture in an organic solvent B, followed by an addition of stannous octanoate as a catalyst; aerating the organic solvent B with nitrogen to remove oxygen; 24-28 h later at a constant temperature of 120-140° C., precipitating a resulting product with excess ether, and drying under vacuum.

8. The method of claim 7, wherein the organic solvent B is anhydrous xylene or toluene.

9. The method of claim 4, wherein combining the amphiphilic tripolymer of P(NIPAM-co-DMAM)-b-PLA and dextran-magnetic layered double hydroxide-fluorouracil comprises: dissolving the dextran-magnetic layered double hydroxide-fluorouracil and the tripolymer in an organic solvent N,N-dimethylformamide; transferring a resulting mixture to a dialysis bag, dialyzing against distilled water and stirring at room temperature.

10. The method of claim 9, wherein the dialysis bag has a molecular-weight cut-off of 8000-14000 g·mol⁻¹.

11. The method of claim 9, wherein a dialysis time is 48 h; the distilled water is renewed every 1 h for first 5 h, and then every 12 h.

12. The method of claim 4, wherein a mass ratio of the dextran-magnetic layered double hydroxide-fluorouracil to the tripolymer is 5-20:20.

13. The method of claim 9, wherein a mass ratio of the dextran-magnetic layered double hydroxide-fluorouracil to the tripolymer is 5-20:20.

14. The method of claim 4, wherein attaching the water-soluble near-infrared CdHgTe quantum dots to the surface layer of the magnetic, thermosensitive micelle comprises:
mixing and grinding the water-soluble near-infrared CdHgTe quantum dots and the magnetic, thermosensitive micelle in a mass ratio of 1-3:1-1 to prepare a mixed powder; and
suspending and dispersing the mixed powder in absolute ethanol thereby yielding a suspension, and ultrasonically dispersing the suspension; separating magnetic particles from the suspension using a magnet, centrifuging, washing a resulting product with absolute ethanol, and drying under vacuum.
